(19) United States
(12) Patent Application Publication
(10) Pub. No.: US 2015/0202382 A1
(43) Pub. Date: Jul. 23, 2015

(54) OPTICAL SENSOR FOR DETECTING INFUSION PUMP CASSETTE

(71) Applicant: Zevex, Inc., Salt Lake City, UT (US)

(72) Inventors: Jeffery T. Juretich, Herriman, UT (US); Michael Marshall, Herriman, UT (US); Michael Elwood, Farmington, UT (US)

(21) Appl. No.: 14/161,825

(22) Filed: Jan. 23, 2014

(51) Int. Cl. A61M 5/50; A61M 5/142; A61M 5/172

(52) U.S. Cl. CPC A61M 5/5086; A61M 5/172; A61M 5/142

(57) ABSTRACT

An infusion pump has an optical detection system for determining whether or not a cassette of an administration tubing set is properly loaded in the pump. Operation of the pump may be enabled or disabled based on a determination of the cassette detection system. The optical cassette detection system includes a light emitter directing a light beam along an optical axis to a corresponding light detector aligned on the optical axis. The cassette detection system also includes a tab carried by the cassette and arranged such that when the cassette is properly loaded in the pump, the tab intersects the optical axis and blocks at least a portion of the light beam from reaching the detector. The detector signal is evaluated by signal evaluation electronics to determine if the detector signal level is below a predetermined threshold, indicating presence of the cassette.

### FIELD OF THE INVENTION

The present invention relates generally to infusion pumps for controlled delivery of liquid food and medications to patients. More specifically, the present invention relates to a sensor system in an infusion pump for detecting the presence or absence of a cassette by which an administration tubing set is operatively connected to the pump.

### BACKGROUND OF THE INVENTION

Programmable infusion pumps are used to carry out controlled delivery of liquid food for enteral feeding and medications for various purposes, for example pain management. In a common arrangement, an infusion pump receives a disposable administration set comprising a cassette removably received by the pump and flexible tubing connected to the cassette for providing a fluid delivery path through the pump.

The cassette itself may be intended for use with a particular infusion pump model or models, and/or with tubing having predetermined properties. In this regard, the cassette may include safety features that are designed and manufactured according to specifications determined at least in part by the intended infusion pump model and/or administration set tubing. The safety features of the cassette may cooperate with corresponding features on the matching pump, and may be manufactured according to size tolerances related to tubing diameter and flexibility. For example, the cassette may have an anti-free flow mechanism for protecting the patient from uncontrolled fluid delivery. The anti-free flow mechanism may take the form of an external pinch clip occluder actuated when the cassette is properly loaded in the pump and a door of the pump is closed. Alternatively, the anti-free flow mechanism may take the form of an internal “in-line occluder” that resides within the flow passage of the tubing, wherein a flow passage is only opened when the cassette is properly loaded in the pump and the pump door is closed.

The cassette may provide additional safety features beyond free flow protection. For example, the cassette may be matched to the pump to maintain a desired volumetric accuracy of the pump, and to ensure correct function of occlusion and air-in-line sensors used to trigger safety alarms.

In view of the safety importance of the cassette, it is desirable to provide means to detect whether or not a matching cassette is loaded in the pump as a precondition to enabling pump operation.

### SUMMARY OF THE INVENTION

In accordance with the present invention, an infusion pump in which an administration set is removably received is provided with an optical detection system for determining whether or not a cassette of the administration set is loaded in the pump. In an embodiment of the present invention, operation of the pump is disabled if a cassette is not loaded in the pump.

The optical cassette detection system includes a light emitter and a corresponding light detector aligned along an optical axis which may extend across a slot in the pump housing. The emitter emits a light beam directed along the optical axis to the detector. The cassette is configured for loading in the pump and includes a tab arranged to block at least a portion of the light beam from reaching the detector when the cassette is properly loaded in the pump. The pump may include a slot between the emitter and the detector configured to receive the tab, such that the tab intersects the optical axis when the cassette is properly loaded in the pump.

The detector signal is evaluated by signal evaluation electronics to determine if the detector signal level is below a predetermined threshold, indicating presence of the cassette. The signal evaluation electronics may be in communication with a pump controller, wherein the pump controller is programmed to disable pump operation unless a cassette is present as determined by the optical cassette detection system.

### BRIEF DESCRIPTION OF THE DRAWINGS

The nature and mode of operation of the present invention will now be more fully described in the following detailed description of the invention taken with the accompanying drawing figures, in which:

FIG. 1 is perspective view of an infusion pump and cassette embodying a cassette detection system in accordance with an embodiment of the present invention;

FIG. 2 is a perspective view of the cassette shown in FIG. 1;

FIG. 3A is a schematic sectional view illustrating a cassette detection system formed in accordance with an embodiment of the present invention, wherein a tab of the cassette is shown prior to insertion into a tab-receiving slot of the pump;

FIG. 3B is a view similar to that of FIG. 3A, however the cassette tab is shown inserted into the slot; and

FIG. 4 is a flow diagram showing decision logic executed by the cassette detection system in accordance with an embodiment of the present invention.

### DETAILED DESCRIPTION OF THE INVENTION

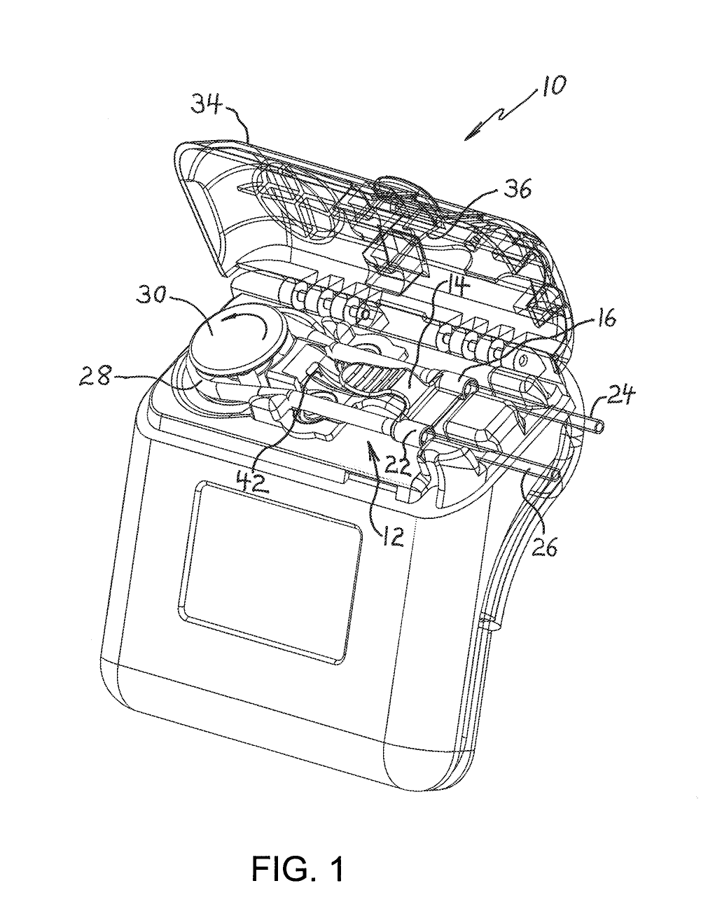

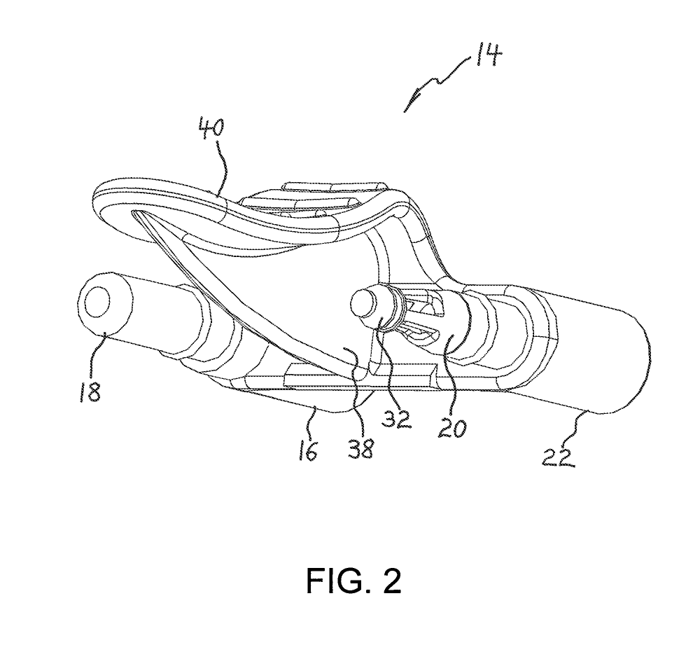

FIG. 1 shows an infusion pump 10 in which an administration set 12 is removably received. Administration set 12 includes a cassette 14, which is shown by itself in FIG. 2. Cassette 14 may include an input connector 16, an upstream loop connector 18 in flow communication with input connector 16, a downstream loop connector 20, and an output connector 22 in flow communication with downstream loop connector 20. Administration set 12 may further include inflow tubing 24 having one end mated to input connector 16 and an opposite end (not shown) connected to a fluid source, and outflow tubing 26 having one end connected to output connector 22 and an opposite end (not shown) connected to a patient. Finally, administration set 14 may further include a pumping segment of tubing 28 having one end mated to upstream loop connector 18 and an opposite end mated to downstream loop connector 20.

In the illustrated embodiment, pump 10 is a rotary peristaltic pump having a rotor 30, wherein pumping segment 28 is wrapped around rotor 30 and is engaged by angularly spaced rollers on rotor 30 as the rotor rotates to provide peristaltic pumping action forcing liquid through the tubing of administration set 12. As may be understood by reference to FIG. 1, when rotor 30 rotates in a counter-clockwise direction, liquid is moved from inflow tubing 24 through input connector 16 and upstream loop connector 18 to pumping segment 28, and then from pumping segment 28 through downstream loop connector 20 and output connector 22 to outflow tubing 26. Although the present invention is described in the context of a rotary peristaltic pump, the invention is not limited to this type of infusion pump. The invention may be practiced with any type of infusion pump that receives an administration set having a cassette.

Cassette 14 may include an in-line occluder 32 which may be incorporated into downstream loop connector 20. In-line occluder 32 prevents flow when pump door 34 is open. An actuator 36 on an underside of pump door 34 engages pumping segment 28 in a manner which opens a flow path around occluder 32 when door 34 is closed.

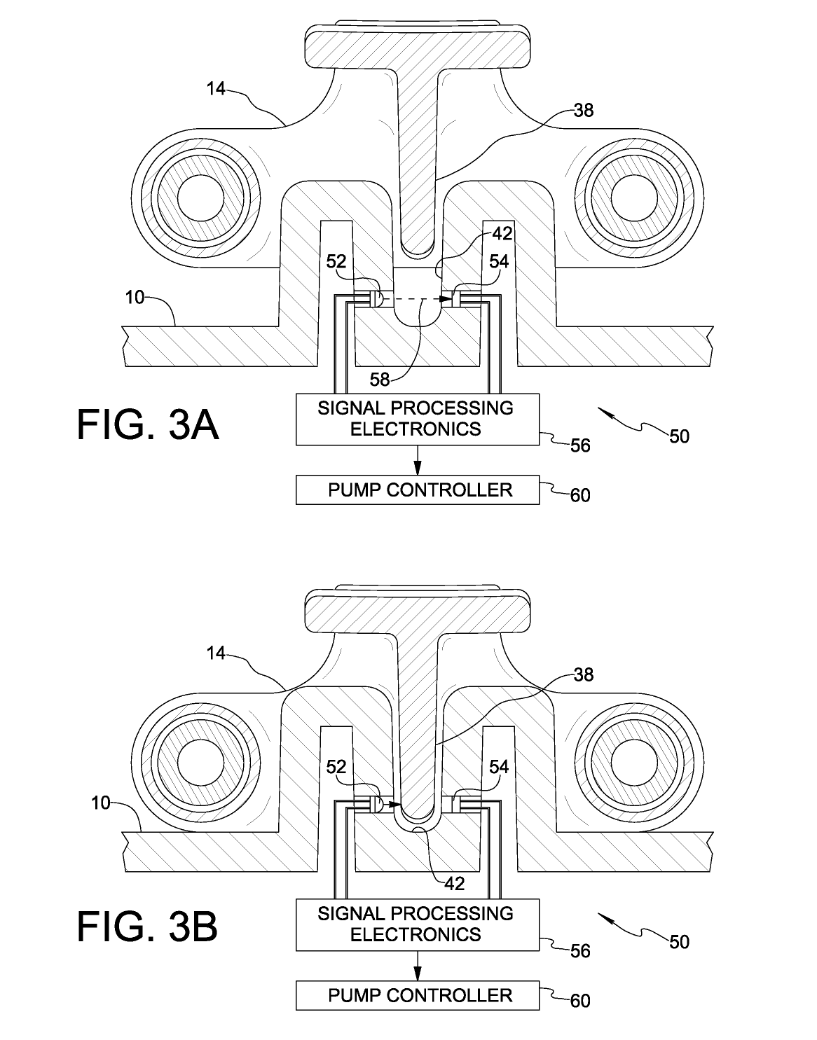

Additional reference is now made to FIGS. 3A and 3B. Cassette 14 includes a tab 38 depending downwardly from a ribbed thumb portion 40 of the cassette. In the present embodiment, tab 38 is a planar tab that is sized for receipt within a corresponding slot 42 in pump 10. Slot 42 may be provided at a location on pump 10 between the upstream and downstream portions of pumping segment 28, and tab 38 may be provided on an underside of thumb portion 40. For example, slot 42 may be midway between the upstream and downstream portions of pumping segment 28 and may be elongated in a direction aligned with the rotation axis of rotor 30, and tab 38 may be midway between one side of cassette 14 having input connector 16 and upstream loop connector 18 and the other side of cassette 14 having downstream loop connector 20 and output connector 22. In this symmetrical arrangement, cassette 14 is easily centered in pump 10 relative to rotor 30 during installation of administration set 12. In an embodiment of the invention, the width of slot 42 is 2.6 mm and the width of tab 38 is 1.7 mm.

As seen in FIGS. 3A and 3B, pump 10 includes an optical cassette detection system 50 operable to detect whether or not cassette tab 38 is present in slot 42.

Cassette detection system 50 includes an optical emitter 52, which may be arranged on one side of slot 42, and a photosensitive detector 54, which may be arranged on an opposite side of slot 42. In the illustrated embodiment, detector 54 is aligned with emitter 52 along an optical axis 58 passing through slot 42. Cassette detection system 50 further includes signal processing electronics 56 connected to photosensitive detector 54 for receiving an electronic signal generated by detector 54 and evaluating the signal. Signal processing electronics 56 may be in communication with a pump controller 60, whereby operation of pump 10 may be controlled based on an evaluation of the detector signal.

In an example embodiment, emitter 52 may be a light-emitting diode (LED) and photosensitive detector 54 may be a photodiode. Emitter 52 and detector 54 may be chosen to operate within a predetermined frequency band, including without limitation a non-visible light frequency band such as an infrared or ultraviolet band. When cassette 14 is not loaded in pump 10, light from emitter 52 travels unimpeded across slot 42 and is received by detector 54. Detector 54 generates a signal, for example a current or voltage signal, having a level corresponding to the intensity of light received thereby. In the unblocked condition shown in FIG. 3A, the detector signal level is above a predetermined threshold. When cassette 14 is loaded in pump 10, as shown in FIG. 3B, tab 38 occupies slot 42. Tab 38 is opaque or substantially opaque, and acts as a shutter or spatial filter to block some or all of the light beam from reaching detector 54 along optical axis 58. Consequently, when cassette 14 is loaded in pump 10, the level of the signal generated by detector 54 drops below the predetermined threshold.

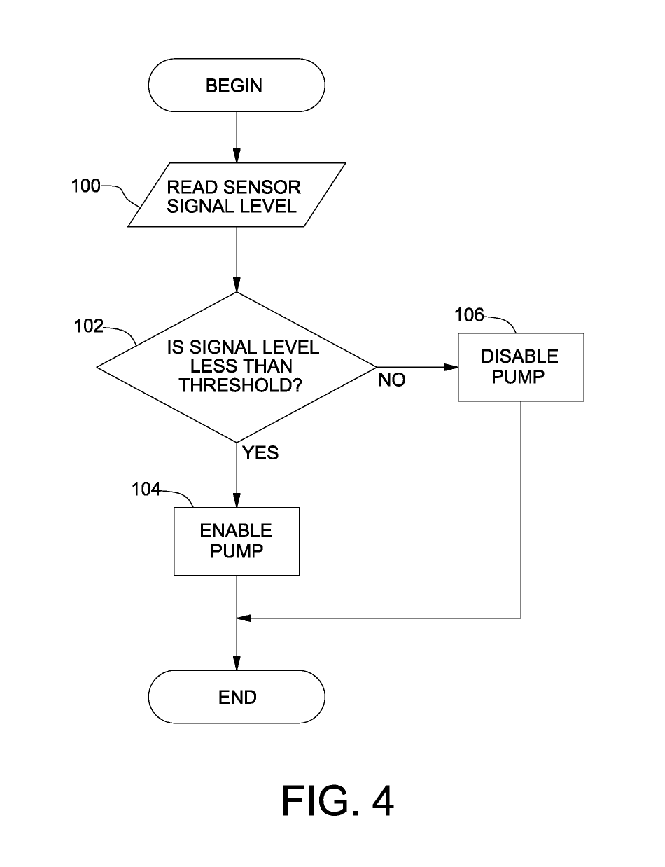

Signal processing electronics 56 evaluates the signal from detector 54 to determine if cassette 14 is loaded in pump 10. The signal processing and evaluation may be completely analog, or the detector signal level may be converted to a digital value and compared to a threshold in a digital comparator circuit. As illustrated in FIG. 4, operation of pump 10 may be enabled or disabled based on the determination made by signal processing electronics 56. In block 100, the level of the detector signal is read. In block 102, the signal level is compared to a predetermined threshold as the basis for a decision. If the signal level is below the threshold, presence of cassette 14 is indicated and flow branches to block 104, wherein pump operation is enabled by pump controller 60. However, if the signal level is above the threshold, flow branches to block 106 and pump operation is disabled by pump controller 60.

Thus, tab 38 on cassette 14 provides a physical feature that alters optical communication between emitter 52 and detector 54 when cassette 14 is properly loaded in pump 10. A wide variety of tab arrangements and optical detection system configurations are of course possible. The centered arrangement of a thin tab 38 on the underside of cassette 14, and the use of a thin slot 42 in pump 10, takes advantage of the tab and slot as a means for guiding and centering the cassette 14 during installation. Moreover, the emitter 52 and detector 54 of cassette detection system 50 are hidden within the pump and are inconspicuous to users. Emitter 52 and detector 54 may be recessed slightly from the surface of slot 42 behind respective transparent barriers to keep dirt and fluid away from the emitter and detector.

While the invention has been described in connection with exemplary embodiments, the detailed description is not intended to limit the scope of the invention to the particular forms set forth. The invention is intended to cover such alternatives, modifications and equivalents of the described embodiment as may be included within the spirit and scope of the invention.

What is claimed is:

1. A system for detecting loading of a cassette in an infusion pump, the system comprising:
an optical emitter mounted to the pump, the optical emitter being arranged to emit a light beam directed along an optical axis;
a photosensitive detector mounted to the pump and arranged to receive the light beam along the optical axis, wherein the photosensitive detector generates a detector signal representing an intensity of light received thereby; and
a tab carried by the cassette, the tab blocking at least a portion of the light beam from reaching the photosensitive detector when the cassette is properly loaded in the pump.

2. The system according to claim 1, further comprising signal processing electronics for evaluating the detector signal to determine whether the cassette is properly loaded in the pump.

3. The system according to claim 1, wherein the tab blocks the entire light beam from reaching the photosensitive detector when the cassette is properly loaded in the pump.

4. The system according to claim 1, wherein the tab blocks a portion of the light beam from reaching the photosensitive detector when the cassette is properly loaded in the pump.

5. The system according to claim 1, wherein the pump includes a slot configured to receive the tab when the cassette is properly loaded in the pump, and the optical emitter and the photosensitive detector are arranged on opposite sides of the slot.

6. An infusion pump comprising:
an optical emitter arranged to emit a light beam directed along an optical axis; and
a photosensitive detector arranged to receive the light beam along the optical axis, wherein the photosensitive detector generates a detector signal representing an intensity of light received thereby;
wherein the detector signal level is decreased from a level above a predetermined threshold to a level below the predetermined threshold by properly loading a cassette in the infusion pump.

7. The infusion pump according to claim 6, further comprising signal processing electronics for evaluating the detector signal to determine whether the cassette is properly loaded in the pump.

8. The infusion pump according to claim 7, further comprising a pump controller connected to the signal processing electronics, wherein the pump controller is programmed to disable pump operation when the signal processing electronics determines that a cassette is not properly loaded in the pump.
